(19) United States
(12) Patent Application Publication
(10) Pub. No.: US 2026/0144457 A1
(43) Pub. Date: May 28, 2026

(54) APPARATUS AND A METHOD FOR PRODUCING INFORMATION INDICATIVE OF CARDIAC ABNORMALITY

(71) Applicant: Precordior Oy, Turku (FI)

(72) Inventors: Juuso BLOMSTER, Turku (FI); Mikko PÄNKÄÄLÄ, Turku (FI); Kamal DEHKORDI, Turku (FI); Tero HURNANEN, Turku (FI)

(21) Appl. No.: 19/430,092

(22) Filed: Dec. 22, 2025

Related U.S. Application Data
(63) Continuation of application No. PCT/EP2025/060934, filed on Apr. 22, 2025.

(30) Foreign Application Priority Data
Apr. 23, 2024 (FI) FI20247058

(51) Int. Cl. A61B 5/11; A61B 5/00

(52) U.S. Cl. CPC A61B 5/1126; A61B 5/6823; A61B 5/7264; A61B 5/7271; A61B 2562/0219

(57) ABSTRACT

Described are computer-implemented methods for determining a cardiac abnormality of a subject. The computer-implemented methods may include obtaining a signal related to a cardiac motion using a non-invasive contact sensor and wherein the signal includes one or more peak measurements of cardiac motion and one or more non-peak measurements of cardiac motion. The computer-implemented methods may include determining one or more peak values relating to the one or more peak measurements and one or more non-peak values relating to the one or more non-peak measurements. The computer-implemented methods may include generating an indication of the cardiac abnormality based at least in part on the one or more peak values and the one or more non-peak values.

### CROSS REFERENCE

This application is a continuation of International Patent Application No. PCT/EP2025/060934, filed Apr. 22, 2025, which claims the benefit of Finnish Patent Application No. FI20247058, filed Apr. 23, 2024, each of which are incorporated by reference herein in their entirety.

### FIELD OF THE DISCLOSURE

The disclosure relates generally to producing information indicative of cardiac abnormality, such as aortic stenosis, heart valve disease, heart failure, atrial fibrillation, and/or other heart conditions. More particularly, the disclosure relates to an apparatus for producing information indicative of cardiac abnormality. Furthermore, the disclosure relates to a method and to a computer program for producing information indicative of cardiac abnormality.

### BACKGROUND

Abnormalities that may occur in the cardiovascular system, if not diagnosed and appropriately treated and/or remedied, may progressively decrease the ability of the cardiovascular system to maintain a blood flow that meets the needs of a body of an individual especially when the individual encounters physical stress. For example, aortic stenosis occurs when the aortic valve narrows, and blood cannot flow normally. Aortic stenosis is typically caused by atherosclerosis, a calcium buildup on the aortic valve over time. These calcium deposits that often come with age make the valve tissue stiff, narrow, and unyielding. A condition of a patient may range from mild to severe. Over time, aortic valve stenosis causes the left ventricle of a heart to pump harder to push blood through the narrowed aortic valve. The extra effort may cause the left ventricle to thicken, enlarge, and weaken. If not diagnosed and appropriately treated and/or remedied, this form of heart valve disease may lead to a heart failure.

Heart failure occurs if the heart cannot pump or fill with blood adequately. Heart failure can be the result of stiffening, thickening, or thinning of the chambers of the heart, or from heart valve malfunctions. Heart failure can also occur from an infection or other disease that damages heart tissue. Atrial fibrillation is an irregular, often rapid heart rate resulting from the atria of the heart beating out of sync with the ventricles. Atrial fibrillation can cause fatigue and can lead to blood clots, stroke, or even death, and can be lifelong.

Cardiovascular imaging techniques are typically used when abnormality of the aortic valve, such as aortic stenosis, is suspected. The cardiovascular imaging techniques include for example the following: transthoracic echocardiogram “TTE”, magnetic resonance imaging “MRI”, cardiac catheterization, transesophageal echocardiogram “TEE”, and computer tomography “CT” scanning. An inherent inconvenience related to imaging techniques of the kind mentioned above is that they typically require expensive equipment and specialized operating personnel. Therefore, there is a need for techniques for producing information indicative of cardiac abnormality, such as aortic stenosis, without a need for expensive equipment and specialized operating personnel.

### SUMMARY

The following presents a simplified summary to provide a basic understanding of some aspects of various invention embodiments. The summary is not an extensive overview of the invention. It is neither intended to identify key or critical elements of the invention nor to delineate the scope of the invention. The following summary merely presents some concepts of the invention in a simplified form as a prelude to a more detailed description of exemplifying embodiments of the invention.

In accordance with the invention, there is provided a new apparatus for producing information indicative of cardiac abnormality, including but not limited to aortic stenosis, heart valve disease, heart failure, and atrial fibrillation. The apparatus according to the invention comprises:
a signal interface for receiving a signal indicative of cardiac motion and measured with a motion sensor, e.g. an accelerometer and/or a gyroscope, having a mechanical contact with a chest of an individual, and
a processing system coupled to the signal interface and configured to determine at least one descriptor value, i.e. one or more descriptor values, from one or more points of the signal indicative of cardiac motion and to send an indicator signal as an output of the apparatus in response to a situation in which the descriptor value or values is/are outside a predetermined value-range.

The processing system is configured to:
form the descriptor value or one of the descriptor values to express strength of highest peaks repeating at a heartbeat rate in the signal relative to strength of other portions of the signal outside the highest peaks, and
set the indicator signal outputted by the apparatus to express presence of cardiac abnormality, e.g. aortic stenosis, in response to a situation in which the descriptor value is outside the predetermined value-range corresponding to healthy cases.

The indicator signal can be transmitted to the individual and can be an instruction to seek treatment and/or advice from a healthcare provider. Alternatively or in combination, the indicator signal can be transmitted to a healthcare provider and can be an indication of the presence of a cardiac abnormality in the individual. The indicator signal may comprise cardiac measurement data transmitted to a healthcare provider.

In the light of empirical data, many cardiac abnormalities increase the relative strength of highest peaks in the signal indicative of cardiac motion with respect to the strength of other portions of the signal outside the highest peaks. For example, in a case of aortic stenosis, an increased pressure difference from the left ventricle to the aorta increases the strength of the highest peaks more strongly than the strength of the other portions of the signal. Thus, the above-mentioned descriptor value can be used as an indicator of cardiac abnormality. The descriptor value can be for example a peak-to-average ratio of the signal, a peak-to-power ratio of the signal, or a peak-to-energy ratio of the signal, or any combination thereof.

The apparatus may comprise a sensor system comprising an accelerometer for measuring the above-mentioned signal indicative of cardiac acceleration and/or a gyroscope for measuring the above-mentioned signal indicative of cardiac rotation. It is also possible that the signal interface is configured to receive the signal from an external apparatus comprising an appropriate sensor system, i.e. it is emphasized that the apparatus does not necessarily comprise means for measuring the signal indicative of the cardiac motion. The apparatus can be for example a smartphone or another hand-held apparatus comprising an accelerometer and/or a gyroscope. A smartphone can be e.g. an Apple iPhone, an Android phone, a Google Pixel phone, Motorola phone, or another type of smartphone. The apparatus can be placed on an individual's chest to measure the above-mentioned signal caused by the motion of the heart. The apparatus can comprise, for example, a patch or wearable sensor able to contact the individual's chest when the individual is laying prone. The apparatus can comprise a medical provider apparatus or other handheld medical apparatus.

In this document, the term “accelerometer” covers sensors of various kinds for measuring acceleration of a linear transverse motion. An accelerometer can be for example a microelectromechanical system “MEMS” based on the law of inertia. In this document, the term “gyroscope” covers sensors of various kinds for measuring angular rotations. A gyroscope can be for example a microelectromechanical system “MEMS” based on an effect of the Coriolis force acting on a back-and-forth turning object inside the MEMS gyroscope.

The above-mentioned value-range corresponding to healthy cases can be determined based on empirical data gathered from a group of patients and/or healthy persons. The limit or limits of the value-range is/are not necessary constant or constants, but the limit or limits can be changing according to an individual under consideration, according to time, and/or according to some other factors. It is also possible to define many value-ranges, each of which represents a specific probability, increased risk factor, or other comparative value indicator of aortic stenosis or some other cardiac abnormality.

In accordance with the invention, there is also provided a new method for producing information indicative of cardiac abnormality, such as aortic stenosis, heart valve disease, heart failure, and/or atrial fibrillation. The method according to the invention comprises:
receiving a signal indicative of cardiac motion and measured with a motion sensor having a mechanical contact with a chest of an individual,
determining at least one descriptor value from one or more points of the signal indicative of cardiac motion, and
outputting an indicator signal in response to a situation in which the descriptor value is outside a predetermined value-range.

In the method according to the invention:
the determining the at least one descriptor value comprises forming the descriptor value or one of the descriptor values to express strength of highest peaks repeating at a heartbeat rate in the signal relative to strength of other portions of the signal outside the highest peaks, and
the indicator signal is set to express presence of cardiac abnormality, e.g. aortic stenosis, in response to a situation in which the descriptor value is outside the predetermined value-range corresponding to healthy cases.

In accordance with the invention, there is also provided a new computer program for producing information indicative of cardiac abnormality, such as aortic stenosis, heart valve disease, heart failure, and/or atrial fibrillation. The computer program comprises computer executable instructions for controlling a programmable processing system to:
receive a signal indicative of cardiac motion and measured with a motion sensor having a mechanical contact with a chest of an individual,
determine at least one descriptor value from one or more points of the signal indicative of cardiac motion, and
output an indicator signal in response to a situation in which the descriptor value is outside a predetermined value-range.

The computer program according to the invention comprises computer executable instructions for controlling the programmable processing system to:
form the descriptor value to express strength of highest peaks repeating at a heartbeat rate in the signal relative to strength of other portions of the signal outside the highest peaks, and
set the indicator signal to express presence of cardiac abnormality, e.g. aortic stenosis, in response to a situation in which the descriptor value is outside the predetermined value-range corresponding to healthy cases.

The indicator signal can express an instruction to contact a healthcare provider. The indicator signal can be transmitted to a healthcare provider to indicate the presence of cardiac abnormality. The indicator signal can be cardiac measurement data transmitted to a healthcare provider.

In accordance with the invention, there is also provided a new computer program product. The computer program product comprises a non-volatile computer readable medium, e.g. a compact disc “CD”, encoded with a computer program according to the invention, a flash drive encoded with a computer program according to the invention, or a digital download of a computer program according to the invention.

A computer readable medium, such as computer-executable code, may take many forms, including but not limited to, a tangible storage medium, a carrier wave medium or physical transmission medium. Non-volatile storage media include, for example, optical or magnetic disks, such as any of the storage apparatuses in any one or more computers or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency “RF” and infrared “IR” data communications. Common forms of computer-readable media therefore include for example: a floppy disk, a flexible disk, a hard disk, a magnetic tape, any other magnetic medium, a compact disc read only memory “CD-ROM”, digital video disc “DVD” or DVD-ROM, any other optical medium, punch cards paper tape, any other physical storage medium with patterns of holes, a random access memory “RAM”, a read only memory “ROM”, a programmable read only memory “PROM”, and an erasable programmable read only memory “EPROM”, a flash-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

Exemplifying and non-limiting embodiments are described in accompanied dependent claims.

Exemplifying and non-limiting embodiments both as to constructions and to methods of operation, together with additional objects and advantages thereof, will be best understood from the following description of specific exemplifying embodiments when read in conjunction with the accompanying drawings.

The verbs “to comprise” and “to include” are used in this document as open limitations that neither exclude nor require the existence of also un-recited features.

The features recited in the accompanied dependent claims are mutually freely combinable unless otherwise explicitly stated.

Furthermore, it is to be understood that the use of “a” or “an”, i.e. a singular form, throughout this document does not exclude a plurality.

The word “predetermined” means adjustably predetermined, advantageously able to be modified in real-time, and advantageously able to be adjusted by an individual whose cardiac condition is under consideration, a healthcare provider, and/or a third party.

### BRIEF DESCRIPTION OF FIGURES

Exemplifying and non-limiting embodiments and their advantages are explained in greater detail below with reference to the accompanying drawings, in which:

FIG. 1 shows a schematic illustration of an apparatus according to an exemplifying and non-limiting embodiment for producing information indicative of cardiac abnormality,

FIG. 2a illustrate a waveform of an exemplifying signal indicative of cardiac acceleration in a normal case,

FIG. 2b illustrate a waveform of an exemplifying signal indicative of cardiac acceleration in a case of aortic stenosis,

FIG. 3a illustrate a waveform of an exemplifying signal indicative of cardiac acceleration in a normal case,

FIG. 3b illustrate a waveform of an exemplifying signal indicative of cardiac acceleration in a case of aortic stenosis,

FIG. 4a illustrate a waveform of an exemplifying signal indicative of cardiac rotation in a normal case,

FIG. 4b illustrate a waveform of an exemplifying signal indicative of cardiac rotation in a case of aortic stenosis, and

FIG. 5 is a flow chart of a method according to an exemplifying and non-limiting embodiment for producing information indicative of cardiac abnormality.

### DESCRIPTION OF EXEMPLIFYING AND NON-LIMITING EMBODIMENTS

The specific examples provided in the description below should not be construed as limiting the scope of the invention. Lists and groups of examples provided in the description are not exhaustive unless otherwise explicitly stated.

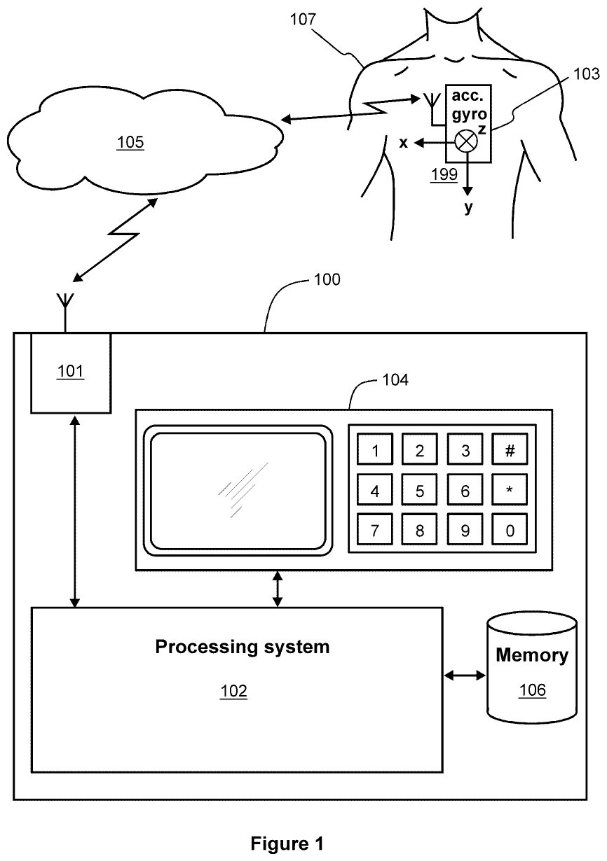

FIG. 1 shows a schematic illustration of an apparatus 100 according to an exemplifying and non-limiting embodiment for producing information indicative of cardiac abnormality, such as aortic stenosis, heart valve disease, heart failure, and/or atrial fibrillation. The apparatus comprises a signal interface 101 for receiving a signal indicative of cardiac motion and measured with a motion sensor having a mechanical contact with a chest of an individual 107. The apparatus 100 comprises a processing system 102 coupled to the signal interface 101. The processing system 102 is configured to:
form a descriptor value describing strength of highest peaks repeating at a heartbeat rate in the signal relative to strength of other portions of the signal outside the highest peaks, and
set an indicator signal outputted by the apparatus to express presence of cardiac abnormality and/or a recommendation to seek a medical provider in response to a situation in which the descriptor value is outside a predetermined value-range corresponding to healthy cases.

The above-mentioned signal is produced with a sensor system 103 that comprises the motion sensor comprising an accelerometer and/or a gyroscope. It is also possible that the sensor system 103 comprises for example an inertial measurement unit “IMU” comprising both an accelerometer and a gyroscope. In the exemplifying situation shown in FIG. 1, the sensor system 103 is placed on the chest of the individual 107. The sensor system 103 can be for example a microelectromechanical system “MEMS”. The temporal duration of the signal can be, for example but not necessarily, between one second and twenty-four hours. In some cases, the temporal duration of the signal can be about less than 1 second, about 1 second, about 2 seconds, about 3 seconds, about 4 seconds, about 5 seconds, about 6 seconds, about 7 seconds, about 8 seconds, about 9 seconds, about 10 seconds, about 20 seconds, about 30 seconds, about 40 seconds, about 50 seconds, about 1 minute, about 2 minutes, about 3 minutes, about 4 minutes, about 5 minutes, about 6 minutes, about 7 minutes, about 8 minutes, about 9 minutes, about 10 minutes, about 15 minutes, about 20 minutes, about 25 minutes, about 30 minutes, about 35 minutes, about 40 minutes, about 45 minutes, about 50 minutes, about 55 minutes, about 1 hour, about 2 hours, about 3 hours, about 4 hours, about 5 hours, about 6 hours, about 7 hours, about 8 hours, about 9 hours, about 10 hours, about 12 hours, about 14 hours, about 16 hours, about 18 hours, about 20 hours, about 22 hours, about 24 hours, about 2 days, about 3 days, about 4 days, about 5 days, about 6 days, about 7 days, or more than about 7 days.

In an exemplifying case where the signal is measured with an accelerometer, the signal is indicative of cardiac acceleration. Correspondingly, in an exemplifying case where the signal is measured with a gyroscope, the signal is indicative of cardiac rotation. It is also possible that the signal has two signal components so that a first signal component is measured with an accelerometer and is indicative of cardiac acceleration and a second signal component is measured with a gyroscope and is indicative of cardiac rotation.

In an apparatus according to an exemplifying and non-limiting embodiment where the signal has the above-mentioned two signal components, the processing system 102 is configured to:
form a first descriptor value describing strength of highest peaks repeating at the heartbeat rate in the first signal component relative to strength of other portions of the first signal component outside the highest peaks,
form a second descriptor value describing strength of highest peaks repeating at the heartbeat rate in the second signal component relative to strength of other portions of the second signal component outside the highest peaks,
set the indicator signal to express presence of cardiac abnormality with a first probability in response to a situation in which at least one of the first and second descriptor values is outside its respective predetermined value-range corresponding to healthy cases, and
set the indicator signal to express presence of cardiac abnormality with a second probability higher than the first probability in response to a situation in which both the first and second descriptor values are outside their predetermined value-ranges corresponding to healthy cases.

The indicator signal outputted by the apparatus 100 can be for example a message shown on a display screen of a user-interface 104. The indicator signal may comprise an instruction to seek treatment and/or advice from a healthcare provider. The apparatus 100 can be configured to transfer the indicator signal to a healthcare provider. Furthermore, cardiac measurement data such as e.g. a recorded waveform of the signal indicative of cardiac motion can be transmitted to the healthcare provider.

In the exemplifying case illustrated in FIG. 1, the sensor system 103 is connected to the signal interface 101 via one or more data transfer links each of which can be for example a radio link or a corded link. The data transfer from the sensor system 103 to the signal interface 101 may take place either directly or via a data transfer network 105 such as e.g. a telecommunication network. In the exemplifying case illustrated in FIG. 1, the sensor system 103 comprises a radio transmitter. It is also possible that the apparatus comprising the processing system 102 is integrated with the sensor system, i.e. the apparatus 100 contains the sensor system. In this exemplifying case, the signal interface is a simple wiring from the sensor system to the processing system. An apparatus comprising an integrated sensor system can be for example a smartphone or another hand-held apparatus which can be placed on the chest of an individual during a measurement phase. The smartphone can be e.g. an Apple iPhone, an Android phone, a Google Pixel phone, Motorola phone, or another type of smartphone. The hand-held apparatus can be for example a patch or wearable sensor able to contact the individual's chest when the individual is lying prone. The hand-held apparatus can be a medical provider apparatus or another handheld medical apparatus.

An apparatus according to an exemplifying and non-limiting embodiment is configured to record the signal indicative of cardiac motion. The recorded signal can be measured within a time window having a fixed temporal start-point and a fixed temporal end-point, or within a sliding time window having a fixed temporal length and moving along with elapsing time. The apparatus may comprise an internal memory 106 for recording the signal and/or the apparatus may comprise a data port for connecting to an external memory. The apparatus may comprise a wireless transceiver for transmitting and receiving data wirelessly to and from an external memory.

There are numerous ways to define and form the descriptor value expressing the strength of the highest peaks of the signal relative to the strength of the other portions of the signal outside the highest peaks. The descriptor value may express for example a peak-to-average ratio of the signal, a peak-to-power ratio of the signal, or a peak-to-energy ratio of the signal. Thus, the invention is not limited to any specific ways to define and form the descriptor value.

In an apparatus according to an exemplifying and non-limiting embodiment, the processing system 102 is configured to form the descriptor value Sacc to be proportional, e.g. directly proportional to the peak-to-energy ratio of the signal:

$${P}_{h} / {\sum }_{i = 1}^{N} ( {q}_{xi}^{2} + {q}_{yi}^{2} + {q}_{zi}^{2} ) , \qquad ( 1 )$$

where i is an index increasing with time, Ph is the peak value i.e. the value of the highest peaks repeating at the heartbeat rate in the signal, N is the number of samples of the signal on a time period under consideration, qxi is an ith sample of an x-component of the cardiac motion, e.g. acceleration or rotation, in a cartesian coordinate system 199, qyi is an ith sample of a y-component of the cardiac motion in the cartesian coordinate system 199, and qzi is an ith sample of a z-component of the cardiac motion in the cartesian coordinate system 199.

The above-mentioned peak value Ph can be defined as:

$$Max \{ \sqrt{( {q}_{xi}^{2} + {q}_{yi}^{2} + {q}_{zi}^{2} )} , i = 1 , 2 , … , N \} , or \qquad ( 2 )$$

$$Max \{ ( {q}_{xi}^{2} + {q}_{yi}^{2} + {q}_{zi}^{2} ) , i = 1 , 2 , … , N \} . \qquad ( 3 )$$

The formula 3 is advantageous in cases where the computation of the square root in formula 2 is wanted to be avoided.

In an apparatus according to an exemplifying and non-limiting embodiment, the processing system 102 is configured to form the descriptor value Sacc to be proportional, e.g. directly proportional to the peak-to-power ratio of the signal:

$${P}_{h} / {\sum }_{i = 1}^{N} ( {q}_{xi}^{2} + {q}_{yi}^{2} + {q}_{zi}^{2} ) / N . \qquad ( 4 )$$

In an apparatus according to an exemplifying and non-limiting embodiment, the processing system 102 is configured to form the descriptor value Sacc to be proportional, e.g. directly proportional to the peak-to-average ratio of the signal:

$${P}_{h} / {\sum }_{i = 1}^{N} \sqrt{( {q}_{xi}^{2} + {q}_{yi}^{2} + {q}_{zi}^{2} )} / N . \qquad ( 5 )$$

In an apparatus according to an exemplifying and non-limiting embodiment, the processing system 102 is configured to form the median value or the arithmetic average of values of the highest peaks belonging to different heartbeat periods, and to use the median value or the arithmetic average as the above-mentioned peak value Ph. The value of the highest peak within a given heartbeat period can be defined according to the above-presented formula 2 or 3 so that samples qxi, qyi, and qzi belonging to the heartbeat period under consideration are used in the formula 2 or 3.

In an apparatus according to an exemplifying and non-limiting embodiment, the processing system 102 is configured to set the indicator signal outputted by the apparatus to express presence of aortic stenosis in response to the situation in which the descriptor value Sacc is outside the predetermined value-range corresponding to the healthy cases.

The value-range of Sacc for healthy cases can be selected to be e.g. a limit value Q and below, i.e. aortic stenosis or other cardiac abnormality is deemed to be present if Sacc>Q, where Q is based on empirical data gathered from a group of patients and healthy persons. The limit value Q is not necessary constant, but the limit value Q can be changing according to an individual under consideration, according to time, and/or according to some other factors. Depending on the accelerometer and/or the gyroscope, the limit value Q can be for example 1, 2, 3, 4, 5, 6, 7, 8, 9, 10, 11, 12, 13, 14, 15, 16, 17, 18, 19, 20, 21, 22, 23, 24, 25, or more than 25. Correspondingly, the value-range of Sacc for healthy cases can be for example 1 and below, 2 and below, 3 and below, 4 and below, 5 and below, 6 and below, 7 and below, 8 and below, 9 and below, 10 and below, 11 and below, 12 and below, 13 and below, 14 and below, 15 and below, 16 and below, 17 and below, 18 and below, 19 and below, 20 and below, 21 and below, 22 and below, 23 and below, 24 and below, 25 and below, or more than 25 and below.

Depending on the accelerometer and/or the gyroscope and on the way of forming the descriptor value Sacc, the limit Q can be a positive value, zero, or a negative value. For example, depending on case, the value-range of the descriptor value Sacc for healthy cases can be zero or above, zero or below, a positive number or above, a positive number or below, a negative number or above, or a negative number or below.

In an apparatus according to an exemplifying and non-limiting embodiment, the processing system 102 is configured to maintain value-ranges each representing a specific probability of cardiac abnormality, e.g. aortic stenosis. The processing system 102 is configured to set, in response to a situation in which the descriptor value belongs to one or more of the value-ranges, the indicator signal to express a highest one of the probabilities of cardiac abnormality related to these one or more of the value-ranges. The value-ranges can be defined for example as follows:
value-range1: Sacc>R1, the probability of aortic stenosis is P1%,
value-range2: Sacc>R2>R1, the probability of aortic stenosis is P2%>P1%,
value-range3: Sacc>R3>R2, the probability of aortic stenosis is P3%>P2%, and
value-range4: Sacc>R4>R3, the probability of aortic stenosis is P4%>P3%.

Each of R1, R2, R3, and R4, can be based on empirical data gathered from a group of patients and healthy persons. Correspondingly, Each of P1, P2, P3, and P4, can be based on empirical data gathered from the group of patients and healthy persons. One or more of the values R1, R2, R3, and R4 is/are not necessarily constant or constants, but the one or more of these values can be changing according to an individual under consideration, according to time, and/or according to some other factors. Depending on the accelerometer and/or on the gyroscope, R1 can be e.g. 1, 2, 3, 4, 5, 6, 7, 8, 9, 10, 11, 12, 13, 14, 15, 16, 17, 18, 19, 20, 21, 22, 23, 24, 25 or more than 25. Correspondingly, R2 can be e.g. R1+1, R1+2, . . . , or R1+more than 25, R3 can be e.g. R2+1, R2+2, . . . , or R2+more than 25, R3 can be e.g. R2+1, R2+2, . . . , or R2+more than 25, and R4 can be e.g. R3+1, R3+2, . . . , or R3+more than 25.

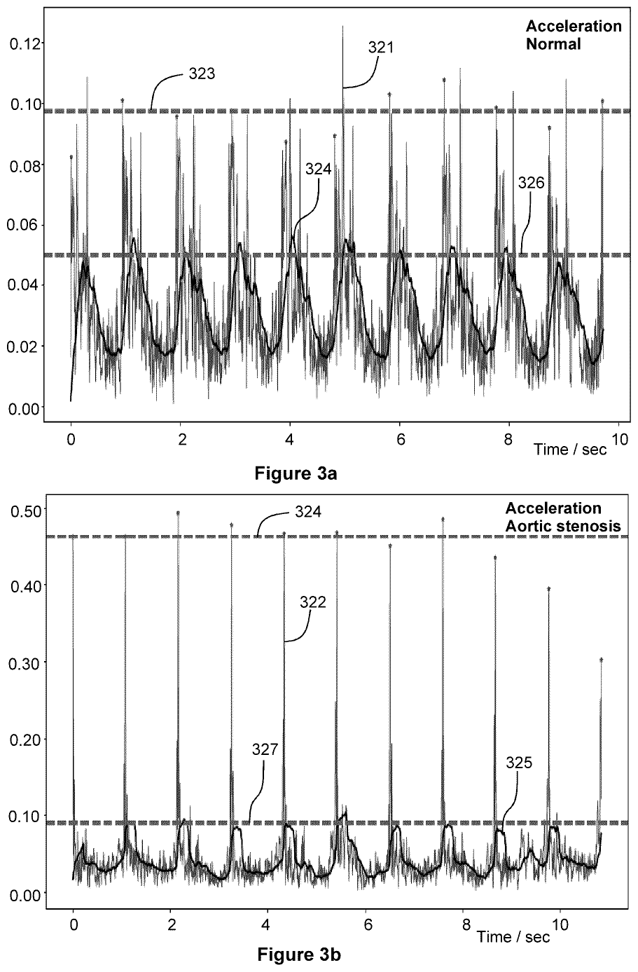

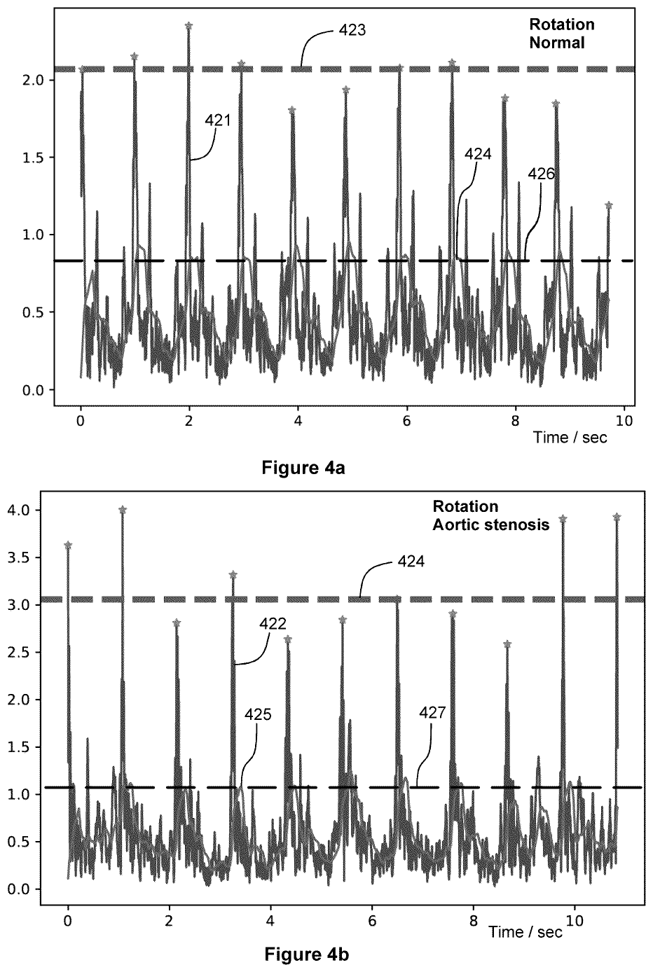

In an apparatus according to an exemplifying and non-limiting embodiment, the processing system 102 is configured to form the descriptor value Sacc to be proportional, e.g. directly proportional to a peak-to-maximum of moving average ratio of the signal. The moving average is the average of the signal within a time-window having a constant temporal length and moving along with time, and the maximum of the moving average is indicative of greatest values repeating at the heartbeat rate in the moving average, i.e. greatest values of the moving average during different heartbeat periods. In an apparatus according to an exemplifying and non-limiting embodiment, the processing system 102 is configured to form a median value of the greatest values repeating at the heartbeat rate in the moving average and to use the median value as the maximum of the moving average when forming the descriptor value. This embodiment is exemplified in the Examples-section of this this document with reference to FIGS. 3a and 3b, and to FIGS. 4a and 4b. In an apparatus according to an exemplifying and non-limiting embodiment, the processing system 102 is configured to form an arithmetic average of the greatest values repeating at the heartbeat rate in the moving average and to use the arithmetic average as the maximum of the moving average when forming the descriptor value.

The processing system 102 can be implemented for example with one or more processor circuits, each of which can be a programmable processor circuit provided with appropriate software, a dedicated hardware processor such as, for example, an application specific integrated circuit “ASIC”, or a configurable hardware processor such as, for example, a field programmable gate array “FPGA”. The memory 106 can be implemented for example with one or more memory circuits, each of which can be e.g. a random-access memory “RAM” apparatus.

An apparatus according to an exemplifying and non-limiting embodiment comprises:
a signal interface for receiving a signal indicative of cardiac motion and measured with a motion sensor having a mechanical contact with a chest of an individual, and
a processing system coupled to the signal interface and configured to determine at least one descriptor value, i.e. one or more descriptor values, from one or more points of the signal indicative of cardiac motion and to send an indicator signal as an output of the apparatus in response to a situation in which the descriptor value or values is/are outside a predetermined value-range.

In an apparatus according to an exemplifying and non-limiting embodiment, the processing system is configured to set the indicator signal to express presence of or a risk of having cardiac abnormality in response to the situation in which the descriptor value or values is/are outside the predetermined value-range.

In an apparatus according to an exemplifying and non-limiting embodiment, the signal interface is further coupled to a sensor system.

In an apparatus according to an exemplifying and non-limiting embodiment, the sensor system comprises an accelerometer, a gyroscope, or both.

In an apparatus according to an exemplifying and non-limiting embodiment, the sensor system is provided in a patch, a wearable sensor, a smartphone, another handheld electronic apparatus, or any combination thereof.

In an apparatus according to an exemplifying and non-limiting embodiment, the signal indicative of cardiac motion is a measure of cardiac acceleration and/or cardiac rotation.

In an apparatus according to an exemplifying and non-limiting embodiment, the one or more descriptor values comprise one or more values of the one or more points of the signal, a relative strength of the one or more points of the signal, an energy ratio of the one or more points of the signal, a power ratio of the one or more points of the signal, an average ratio of the one or more points of the signal, or any combination thereof.

In an apparatus according to an exemplifying and non-limiting embodiment, the indicator signal is transmitted to the individual and/or to a healthcare provider of the individual.

In an apparatus according to an exemplifying and non-limiting embodiment, the indicator signal is an instruction to seek treatment from a healthcare provider, instruction to seek advice from a healthcare provider, an indication of the presence of cardiac abnormality of the individual, cardiac measurement data, or any combination thereof.

In an apparatus according to an exemplifying and non-limiting embodiment, the cardiac abnormality comprises aortic stenosis, heart valve disease, heart failure, or atrial fibrillation, or any combination thereof.

In an apparatus according to an exemplifying and non-limiting embodiment, the indicator signal indicates presence of aortic stenosis.

In an apparatus according to an exemplifying and non-limiting embodiment, the signal interface and the processing system are provided in a same electronic device.

In an apparatus according to an exemplifying and non-limiting embodiment, the signal interface and the processing system are provided in different electronic devices that are in communication with each other.

An apparatus according to an exemplifying and non-limiting embodiment comprises:
a signal interface configured to receive acceleration of a chest of an individual measured with a sensor system placed on the chest of the individual,
a processing system configured to calculate one or more parameters related to a cardiac abnormality from the measured acceleration of the chest of the individual and to detect a presence of or a risk of having cardiac abnormality based on the calculated one or more parameters, wherein the cardiac abnormality comprises aortic stenosis.

In an apparatus according to an exemplifying and non-limiting embodiment, the processing system is configured to calculate the one or more parameters so that the calculation of the one or more parameters further comprises determining one or more descriptor values from the measured acceleration, wherein the one or more descriptor values comprise one or more values of one or more points of the measured acceleration, a relative strength of the one or more points of the measured acceleration, an energy ratio of the one or more points of the measured acceleration, a power ratio of the one or more points of the measured acceleration, an average ratio of the one or more points of the measured acceleration, or any combination thereof. The processing system can be configured to output an indicator signal expressing that the one or more descriptor values is/are outside a value-range that can be a fixed or adjustable value-range. The indicator signal can be transmitted to the individual and/or to a healthcare provider of the individual. The indicator signal may comprise one or more instructions to seek treatment from a healthcare provider, one or more instructions to seek advice from a healthcare provider, an indication of the presence of cardiac abnormality of the individual, or cardiac measurement data, or any combination thereof.

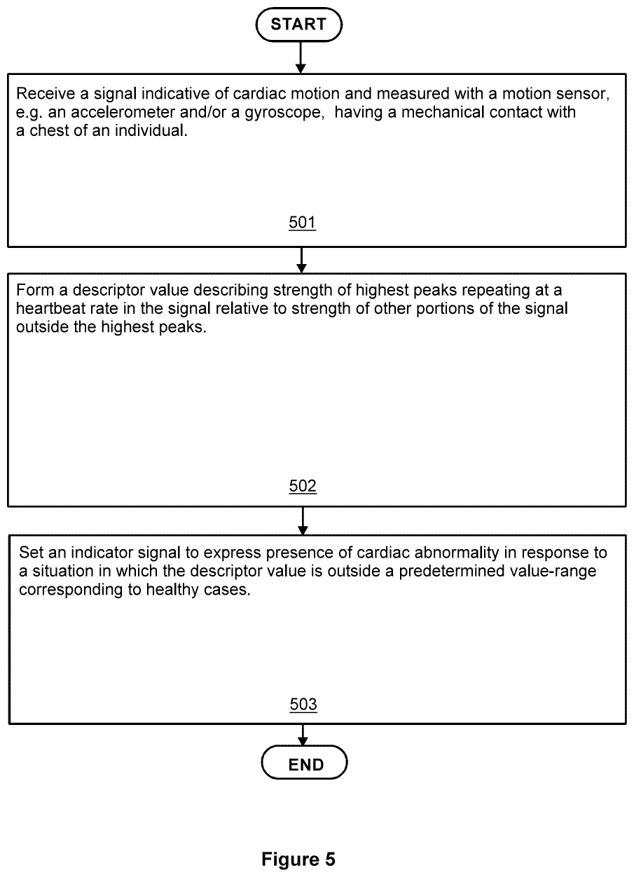

FIG. 5 shows a flow chart of a method according to an exemplifying and non-limiting embodiment for producing information indicative of cardiac abnormality, such as aortic stenosis, heart valve disease, heart failure, and/or atrial fibrillation. The method comprises the following actions:
action 501: receiving a signal indicative of cardiac motion and measured with a motion sensor, e.g. an accelerometer and/or a gyroscope, having a mechanical contact with a chest of an individual,
action 502: forming a descriptor value describing strength of highest peaks repeating at a heartbeat rate in the signal relative to strength of other portions of the signal outside the highest peaks, and
action 503: setting an indicator signal to express presence of cardiac abnormality in response to a situation in which the descriptor value is outside a predetermined value-range corresponding to healthy cases.

A method according to an exemplifying and non-limiting embodiment comprises measuring the above-mentioned signal with the motion sensor from the chest of the individual. A method according to another exemplifying and non-limiting embodiment comprises reading this signal from a memory, in which case the signal has been measured earlier and recorded in the memory. A method according to an exemplifying and non-limiting embodiment comprises receiving the signal from an external data transfer system. Therefore, the measuring is not an essential and necessary step of methods according to embodiments of the invention.

In a method according to an exemplifying and non-limiting embodiment, the signal is indicative of cardiac acceleration and the signal is measured with an accelerometer having a mechanical contact with the chest of the individual.

In a method according to an exemplifying and non-limiting embodiment, the signal is indicative of cardiac rotation and the signal is measured with a gyroscope having a mechanical contact with the chest of the individual.

In a method according to an exemplifying and non-limiting embodiment, the descriptor value is proportional to a peak-to-average ratio of the signal.

In a method according to an exemplifying and non-limiting embodiment, the descriptor value is proportional to a peak-to-power ratio of the signal.

In a method according to an exemplifying and non-limiting embodiment, the descriptor value is proportional to a peak-to-energy ratio of the signal.

In a method according to an exemplifying and non-limiting embodiment, the descriptor value is proportional to a peak-to-maximum of moving average ratio of the signal. The moving average is an average of the signal within a time-window having a constant temporal length and moving along with time, and the maximum of the moving average is indicative of greatest values repeating at the heartbeat rate in the moving average.

A method according to an exemplifying and non-limiting embodiment comprises forming a median value of the greatest values repeating at the heartbeat rate in the moving average and to use the median value as the above-mentioned maximum of the moving average when forming the descriptor value.

A method according to an exemplifying and non-limiting embodiment comprises forming an arithmetic average of the greatest values repeating at the heartbeat rate in the moving average and to use the arithmetic average as the above-mentioned maximum of the moving average when forming the descriptor value.

A method according to an exemplifying and non-limiting embodiment comprises forming a median value of values of the highest peaks and using the median value as a peak value expressing the strength of the highest peaks when forming the descriptor value.

A method according to an exemplifying and non-limiting embodiment comprises forming an arithmetic average of values of the highest peaks and using the arithmetic average as a peak value expressing the strength of the highest peaks when forming the descriptor value.

A method according to an exemplifying and non-limiting embodiment comprises setting the indicator signal to express presence of aortic stenosis in response to the situation in which the descriptor value is outside the predetermined value-range corresponding to the healthy cases.

A method according to an exemplifying and non-limiting embodiment comprises maintaining value-ranges each representing a specific probability of cardiac abnormality, e.g. aortic stenosis. The method comprises setting, in response to a situation in which the descriptor value belongs to one or more of the value-ranges, the indicator signal to express a highest one of the probabilities of cardiac abnormality related to these one or more of the value-ranges.

A method according to an exemplifying and non-limiting embodiment comprises:
receiving a signal indicative of cardiac motion and measured with a motion sensor having a mechanical contact with a chest of an individual,
determining at least one descriptor value, i.e. one or more descriptor value, from one or more points of the signal indicative of cardiac motion, and
outputting an indicator signal in response to a situation in which the descriptor value or values is/are outside a predetermined value-range.

A method according to an exemplifying and non-limiting embodiment is a computer-implemented method.

A method according to an exemplifying and non-limiting embodiment comprises setting the indicator signal to express presence of or a risk of having cardiac abnormality in response to the situation in which the descriptor value or values is/are outside the predetermined value-range.

In a method according to an exemplifying and non-limiting embodiment, the signal indicative of cardiac motion is measured with a sensor system.

In a method according to an exemplifying and non-limiting embodiment, the sensor system is provided in a patch, a wearable sensor, an interface of a smartphone or other handheld electronic device, or any combination thereof.

In a method according to an exemplifying and non-limiting embodiment, the sensor system comprises a gyroscope, an accelerometer, or both.

In a method according to an exemplifying and non-limiting embodiment, the signal indicative of cardiac motion is a signal indicative of movement of the chest of the individual.

In a method according to an exemplifying and non-limiting embodiment, the movement of the chest of the individual comprises movement indicative of cardiac acceleration.

In a method according to an exemplifying and non-limiting embodiment, the one or more descriptor values comprise one or more values of the one or more points of the signal, a relative strength of the one or more points of the signal, an energy ratio of the one or more points of the signal, a power ratio of the one or more points of the signal, an average ratio of the one or more points of the signal, or any combination thereof.

In a method according to an exemplifying and non-limiting embodiment, the indicator signal is transmitted to the individual and/or to a healthcare provider of the individua.

In a method according to an exemplifying and non-limiting embodiment, the indicator signal comprises one or more instructions to seek treatment from a healthcare provider, one or more instructions to seek advice from a healthcare provider, an indication of the presence of cardiac abnormality of the individual, cardiac measurement data, or any combination thereof.

In a method according to an exemplifying and non-limiting embodiment, the cardiac abnormality comprises aortic stenosis, heart valve disease, heart failure, atrial fibrillation, or any combination thereof.

In a method according to an exemplifying and non-limiting embodiment, the indicator signal indicates presence of aortic stenosis.

A method according to an exemplifying and non-limiting embodiment comprises:
measuring, with a sensor system placed on a chest of an individual, acceleration of the chest of the individual,
calculating one or more parameters related to a cardiac abnormality from the measured acceleration of the chest of the individual, and
detecting presence of or a risk of having the cardiac abnormality based on the calculated one or more parameters, wherein the cardiac abnormality comprises aortic stenosis.

In a method according to an exemplifying and non-limiting embodiment, the calculating the one or more parameters further comprises determining one or more descriptor values from the measured acceleration, wherein the one or more descriptor values comprise one or more values of one or more points of the measured acceleration, a relative strength of the one or more points of the measured acceleration, an energy ratio of the one or more points of the measured acceleration, a power ratio of the one or more points of the measured acceleration, an average ratio of the one or more points of the measured acceleration, or any combination thereof. The computer-implemented method may further comprise outputting an indicator signal expressing that the one or more descriptor values is/are outside a value-range that can be a fixed or adjustable value-range. The indicator signal can be transmitted to the individual and/or to a healthcare provider of the individual. The indicator signal may comprise one or more instructions to seek treatment from a healthcare provider, one or more instructions to seek advice from a healthcare provider, an indication of the presence of cardiac abnormality of the individual, cardiac measurement data, or any combination thereof.

A computer program according to an exemplifying and non-limiting embodiment comprises computer executable instructions for controlling a programmable processing system to carry out a method according to any of the above-presented exemplifying and non-limiting embodiments.

A computer program according to an exemplifying and non-limiting embodiment comprises software modules for producing information indicative of cardiac abnormality, such as aortic stenosis, heart valve disease, heart failure, atrial fibrillation, and/or other cardiac abnormality. The software modules comprise computer executable instructions for controlling a programmable processing system to:
receive a signal indicative of cardiac motion measured with a motion sensor, e.g. an accelerometer and/or a gyroscope, having a mechanical contact with a chest of an individual,
form a descriptor value describing strength of highest peaks repeating at a heartbeat rate in the signal relative to strength of other portions of the signal outside the highest peaks, and
set an indicator signal to express presence of cardiac abnormality in response to a situation in which the descriptor value is outside a predetermined value-range corresponding to healthy cases.

The software modules can be e.g. subroutines or functions implemented with a suitable programming language and with a compiler suitable for the programming language and for the programmable processing system under consideration. It is worth noting that a source code corresponding to a suitable programming language represents the computer executable software modules because the source code contains the information needed for controlling the programmable processing system to carry out the above-presented actions and compiling changes only the format of the information. Furthermore, it is also possible that the programmable processing system is provided with an interpreter so that a source code implemented with a suitable programming language does not need to be compiled prior to running.

A computer program product according to an exemplifying and non-limiting embodiment comprises a computer readable medium, e.g. a compact disc “CD”, encoded with a computer program according to an embodiment of the invention, a flash drive encoded with a computer program according to the invention, or a digital download of a computer program according to the invention.

A computer readable medium according to an exemplifying and non-limiting embodiment is encoded with a computer program according to an embodiment of invention.

A computer readable medium, such as computer-executable code, may take many forms, including but not limited to, a tangible storage medium, a carrier wave medium or physical transmission medium. Non-volatile storage media include, for example, optical or magnetic disks, such as any of the storage apparatuses in any one or more computers or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency “RF” and infrared “IR” data communications. Common forms of computer-readable media therefore include for example: a floppy disk, a flexible disk, hard disk, magnetic tape, any other magnetic medium, a compact disc read only memory “CD-ROM”, digital video disc “DVD” or DVD-ROM, any other optical medium, punch cards paper tape, any other physical storage medium with patterns of holes, a random access memory “RAM”, a read only memory “ROM”, a programmable read only memory “PROM” and an erasable programmable read only memory “EPROM”, a flash-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

A signal according to an exemplifying and non-limiting embodiment is encoded to carry information defining a computer program according to an embodiment of invention.

### EXAMPLES

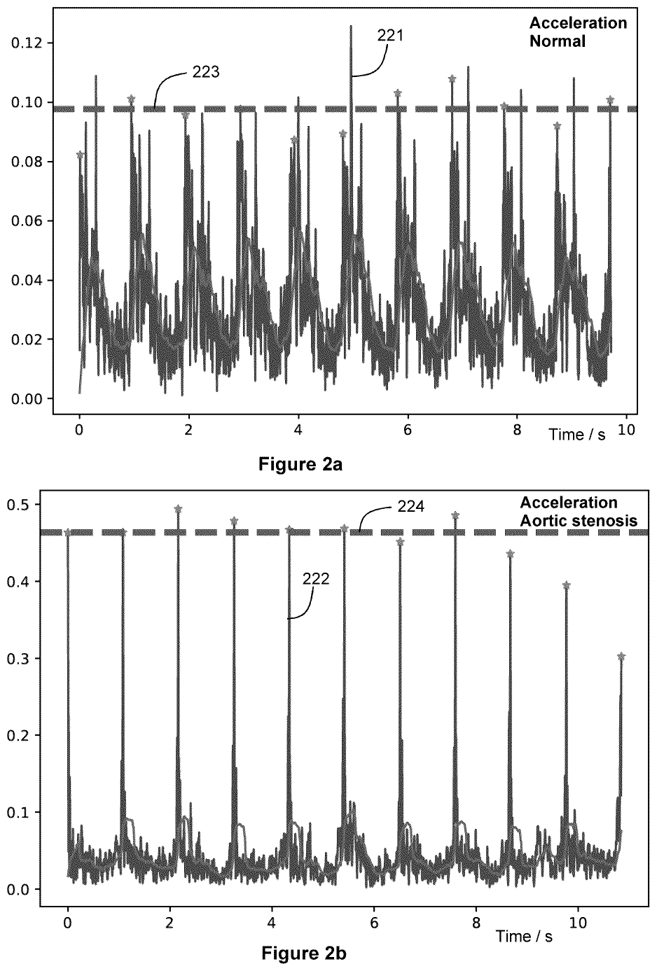

FIG. 2a illustrates a waveform of an exemplifying signal 221 indicative of cardiac acceleration in a normal case. Correspondingly, FIG. 2b illustrates a waveform of an exemplifying signal 222 indicative of cardiac acceleration in a case of aortic stenosis.

The signal 221 shown in FIG. 2a is measured with a three-axis accelerometer and the signal 221 at a time moment ti is defined as:

$$\sqrt{( {a}_{xi}^{2}} + {a}_{yi}^{2} + {a}_{zi}^{2} ) , \qquad ( 6 )$$

where i is an index increasing with time, axi is an ith sample of an x-component of the signal 221, i.e. an x-directional component of the cardiac acceleration, in a cartesian coordinate system, a_{yi} is an ith sample of a y-component of the signal 221 in the cartesian coordinate system, and a_{zi} is an ith sample of a z-component of the signal 221 in the cartesian coordinate system. The z-direction is towards the chest of the individual as illustrated with the coordinate system 199 in FIG. 1. Correspondingly, the signal 222 shown in FIG. 2b is measured with a three-axis accelerometer and is defined according to the above-presented formula 6.

In FIGS. 2a and 2b, dashed lines 223 and 224 denote the median values of peak values of successive heart-beat periods, respectively. As discussed earlier in this document, these median values can be used as the peak value Ph when forming the descriptor value Sacc. The median values are advantageous since they are tolerant against noise and outliers.

In the exemplifying normal case shown in FIG. 2a, the median value i.e. the peak value Ph is about 0.099 and the average of the signal 221 is about 0.05. Thus, the peak-to-average ratio according to the above-presented formula 5 is about 1.98.

In the exemplifying aortic stenosis case shown in FIG. 2b, the median value i.e. the peak value Ph is about 0.46 and the average of the signal 222 is about 0.08. Thus, the peak-to-average ratio according to the above-presented formula 5 is about 5.75.

In the exemplifying cases illustrated in FIGS. 2a and 2b, the value-range of Sacc for healthy cases can be selected to be e.g. 4 and below, i.e. aortic stenosis is deemed to be present if Sacc>4.

In some embodiments, the lower limit of the Sacc value-range on which cardiac abnormality, e.g. aortic stenosis, is deemed to be present can be between 0.5 and 10.0. In some embodiments, the Sacc value-range on which cardiac abnormality, e.g. aortic stenosis, is deemed to be present can be >0.1, >0.5, >1.0, >1.5, >2.0, >2.5, >3.0, >3.5, >4.0, >4.5, >5.0, >5.5, >6.0, >6.5, >7.0, >7.5, >8.0, >8.5, >9.0, >9.5, or >10.0.

As mentioned earlier in this document, one can define value-ranges each representing a specific probability of cardiac abnormality, e.g. aortic stenosis, and, in response to a situation in which the descriptor value belongs to one or more of the value-ranges, the indicator signal can be set to express a highest one of the probabilities of cardiac abnormality related to these one or more of the value-ranges. In the exemplifying cases illustrated in FIGS. 2a and 2b, the value-ranges can be defined for example as follows:
value-range1: Sacc>3.5, the probability of aortic stenosis is P1%,
value-range2: Sacc>4.0, the probability of aortic stenosis is P2%>P1%,
value-range3: Sacc>4.5, the probability of aortic stenosis is P3%>P2%, and
value-range4: Sacc>5.5, the probability of aortic stenosis is P4%>P3%.

FIG. 3a illustrates a waveform of an exemplifying signal 321 indicative of cardiac acceleration in a normal case. Correspondingly, FIG. 3b illustrates a waveform of an exemplifying signal 322 indicative of cardiac acceleration in a case of aortic stenosis.

The signal 321 shown in FIG. 3a is measured with a three-axis accelerometer and is defined according to the above-presented formula 6. Correspondingly, the signal 322 shown in FIG. 3b is measured with a three-axis accelerometer and is defined according to the above-presented formula 6. In FIGS. 3a and 3b, dashed lines 323 and 324 denote the median values of peak values of successive heart-beat periods, respectively. As discussed above, these median values can be used as the above-mentioned peak value Ph when forming the descriptor value Sacc. Waveforms 324 and 325 illustrate moving averages of the signals 321 and 322, respectively. In this exemplifying case, each of the moving averages is an arithmetic average of 45 most recent samples of the respective signal but it is also possible to use a different number of samples in the moving averages. Dashed lines 326 and 327 denote the median values of the greatest values of the moving averages in successive heart-beat periods.

In the exemplifying normal case shown in FIG. 3a, the median value of the peak values of the successive heart-beat periods, i.e. the peak value Ph, is about 0.099 and the median value of the greatest values of the moving average in the successive heart-beat periods is about 0.05. Thus, the peak-to-maximum of moving average ratio is about 1.98.

In the exemplifying aortic stenosis case shown in FIG. 3b, the median value of the peak values of the successive heart-beat periods, i.e. the peak value Ph, is about 0.46 and the median value of the greatest values of the moving average in the successive heart-beat periods is about 0.09. Thus, the peak-to-maximum of moving average ratio is about 5.11.

In the exemplifying cases illustrated in FIGS. 3a and 3b, the value-range of Sacc, i.e. the peak-to-maximum of moving average ratio, for healthy cases can be selected to be e.g. 4 and below, i.e. aortic stenosis is deemed to be present if Sacc>4.

FIG. 4a illustrates a waveform of an exemplifying signal 421 indicative of cardiac rotation in a normal case. Correspondingly, FIG. 4b illustrates a waveform of an exemplifying signal 422 indicative of cardiac rotation in a case of aortic stenosis.

The signal 421 shown in FIG. 4a is measured with a three-axis gyroscope and the signal 421 at a time moment ti is defined as:

$$\sqrt{( {ω}_{xi}^{2}} + {ω}_{yi}^{2} + {ω}_{zi}^{2} ) , \qquad ( 7 )$$

where i is an index increasing with time, □xi is an ith sample of an x-component of the signal 421 in a cartesian coordinate system, i.e. cardiac rotation with respect to the x-direction of the cartesian coordinate system, □yi is an ith sample of a y-component of the signal 421 in the cartesian coordinate system, and □zi is an ith sample of a z-component of the signal in the cartesian coordinate system. The z-direction is towards the chest of the individual as illustrated with the coordinate system 199 in FIG. 1. Correspondingly, the signal 422 shown in FIG. 4b is measured with a three-axis gyroscope and is defined according to the above-presented formula 7.

In FIGS. 4a and 4b, dashed lines 423 and 424 denote the median values of peak values of successive heart-beat periods, respectively. As discussed above, these median values can be used as the above-mentioned peak value Ph when forming the descriptor value Sacc. The median values are advantageous since they are tolerant against noise and outliers.

Waveforms 424 and 425 illustrate moving averages of the signals 421 and 422, respectively. In this exemplifying case, each of the moving averages is an arithmetic average of 45 most recent samples of the respective signal but it is also possible to use a different number of samples in the moving averages. Dashed lines 426 and 427 denote the median values of the greatest values of the moving averages in successive heart-beat periods.

In the exemplifying normal case shown in FIG. 4a, the median value of the peak values of the successive heart-beat periods, i.e. the peak value Ph, is about 2.1 and the median value of the greatest values of the moving average in the successive heart-beat periods is about 0.8. Thus, the peak-to-maximum of moving average ratio is about 2.6.

In the exemplifying aortic stenosis case shown in FIG. 4b, the median value of the peak values of the successive heart-beat periods, i.e. the peak value Ph, is about 3.05 and the median value of the greatest values of the moving average in the successive heart-beat periods is about 1.1. Thus, the peak-to-maximum of moving average ratio is about 2.8.

In the exemplifying cases illustrated in FIGS. 4a and 4b, the value-range of Sacc, i.e. the peak-to-maximum of moving average ratio, for healthy cases can be selected to be e.g. 2.7 and below, i.e. aortic stenosis is deemed to be present if Sacc>2.7.

### REMARKS

The specific examples provided in the description given above should not be construed as limiting the scope of the invention. Lists and groups of examples provided in the description given above are not exhaustive unless otherwise explicitly stated.

What is claimed is:

1. A computer-implemented method for determining a cardiac abnormality of a subject, the computer-implemented method comprising:
(a) obtaining a signal related to a cardiac motion, wherein the signal is generated using a non-invasive contact sensor, and wherein the signal comprises one or more peak measurements of cardiac motion and one or more non-peak measurements of cardiac motion;
(b) determining (i) one or more peak values relating to the one or more peak measurements and (ii) one or more non-peak values relating to the one or more non-peak measurements; and
(c) generating an indication of the cardiac abnormality based at least in part on the one or more peak values and the one or more non-peak values.

2. The computer-implemented method of claim 1, wherein the signal comprises a cardiac rotation signal.

3. The computer-implemented method of claim 1, wherein the signal comprises a cardiac acceleration signal.

4. The computer-implemented method of claim 1, wherein determining the one or more peak values further comprises comparing the one or more peak measurements to an average value of the signal.

5. The computer-implemented method of claim 4, further comprising determining one or more descriptor values based at least in part on comparing the one or more peak values to the one or more non-peak values.

6. The computer-implemented method of claim 5, wherein determining the one or more descriptor values further comprises determining a difference between signal energy of the one or more peak values and signal energy of the one or more non-peak values.

7. The computer-implemented method of claim 5, wherein determining the one or more descriptor values further comprises determining a peak-to-average ratio of the signal, a peak-to-power ratio of the signal, or a peak-to-energy ratio of the signal, or any combination thereof.

8. The computer-implemented method of claim 7, wherein the one or more descriptor values are proportional to one or more of: the peak-to-average ratio of the signal, the peak-to-power ratio of the signal, or the peak-to-energy ratio of the signal, or any combination thereof.

9. The computer-implemented method of claim 8, further comprising determining the cardiac abnormality based at least in part on a comparison of the one or more descriptor values to a value range.

10. The computer-implemented method of claim 9, wherein generating the indication of the cardiac abnormality further comprises determining the one or more descriptor values to be outside the value range.

11. The computer-implemented method of claim 1, wherein obtaining the signal related to the cardiac motion further comprises measuring a cardiac rotation of the subject using the non-invasive contact sensor.

12. The computer-implemented method of claim 11, wherein measuring the cardiac rotation of the subject further comprises contacting a chest of the subject with the non-invasive contact sensor.

13. The computer-implemented method of claim 11, wherein the non-invasive contact sensor comprises a gyroscope, or an accelerometer, or both.

14. The computer-implemented method of claim 1, wherein obtaining the signal related to the cardiac motion further comprises measuring a cardiac acceleration of the subject using the non-invasive contact sensor.

15. The computer-implemented method of claim 14, wherein measuring the cardiac acceleration of the subject further comprises contacting a chest of the subject with the non-invasive contact sensor.

16. The computer-implemented method of claim 14, wherein the non-invasive contact sensor comprises a gyroscope, or an accelerometer, or both.

17. The computer-implemented method of claim 16, wherein the non-invasive contact sensor comprises a sensor system.

18. The computer-implemented method of claim 1, wherein the cardiac abnormality comprises aortic stenosis, heart valve disease, heart failure, or atrial fibrillation, or any combination thereof.

19. The computer-implemented method of claim 18, wherein the cardiac abnormality is aortic stenosis.

20. A system for determining a cardiac abnormality in a subject, the system comprising a memory and one or more processors,
wherein the memory is configured to store machine-executable instructions that, upon execution by the one or more processors, causes the one or more processors to:
(a) obtain a signal related to cardiac motion of the subject, wherein the signal is generated using a non-invasive contact sensor, and wherein the signal comprises one or more peak measurements of cardiac motion and one or more non-peak measurements of cardiac motion;
(b) determine (i) one or more peak values relating to the one or more peak measurements and (ii) one or more non-peak values relating to the one or more non-peak measurements; and
(c) generate an indication of the cardiac abnormality based at least in part on the one or more peak values and the one or more non-peak values.
